(19) United States
(12) Patent Application Publication
(10) Pub. No.: US 2019/0099530 A1
(43) Pub. Date: Apr. 4, 2019

(54) Device for Storing Blood and Method for Use Thereof

(71) Applicant: Haemair Limited, Swansea South Wales (GB)

(72) Inventors: William Richard Johns, Swansea South Wales (GB); Alan Frederick Evans, Swansea South Wales (GB); Ronald Kelvin Knight, Swansea South Wales (GB)

(21) Appl. No.: 16/087,028

(22) PCT Filed: Mar. 17, 2017
(86) PCT No.: PCT/GB2017/050742

§ 371 Date: Sep. 20, 2018

(30) Foreign Application Priority Data
Mar. 21, 2016 (GB) 1604737.5

(51) Int. Cl. A61M 1/02; A61J 1/14; A61J 1/10

(52) U.S. Cl. CPC A61M 1/0259; A61J 1/1475; A01N 1/021; A61M 1/0272; A61M 1/0209; A61J 1/10

(57) ABSTRACT

A mass exchanger for use in storing blood or a blood product such as packed cells is described. The mass exchanger comprises an external casing, a cavity is provided within the casing having a region for storing blood and one or more channels extending within the casing for accommodating flow of a treatment fluid, the one or more channels each being at least partly bounded by a permeable membrane to allow transfer of chemical species between the channel and the cavity; wherein the casing comprises at least one flexible wall. The mass exchanger can include a region for storing blood comprising a bag. Here the bag or the casing comprise at least one flexible wall. A method of storing blood in the mass exchanger is also described.

### CROSS-REFERENCE TO RELATED APPLICATIONS

This application is a national stage application under 35 U.S.C. § 371 of International Patent Application no. PCT/GB2017/050742, filed Mar. 17, 2017 which claims the benefit of priority of United Kingdom Patent Application no. 1604737.5, filed Mar. 21, 2016.

### FIELD OF THE INVENTION

The present application relates to devices for blood storage, in particular to devices that allow the chemical composition of the blood to be controlled, and to methods for use thereof,

### BACKGROUND OF THE INVENTION

A variety of methods have been proposed in order to extend the storage life of transfusion blood and blood derivatives (such as packed cells). Most of these methods require 3 stages of treatment, followed by transfer to a blood bag for transfusion to the patient. These stages are:

1) Treatment at, or soon after, the point of donation—for example to achieve very low oxygen and carbon dioxide concentrations (as described, for example, in WO 2011/014855, WO 2011/046841, WO 2012/027582);

2) Storage using special conditions to avoid reversal of the initial treatment (occurring, for example, through slow diffusion of oxygen and/or carbon dioxide back into the blood or blood product). This may involve storing blood below 0° C., as described, for example, in EP0371178;

3) Reversal of the initial treatment, such as addition of the species removed or removal of preservatives added to the blood.

To avoid cross-contamination, the apparatus used at each of these steps is a single-use disposable item. Furthermore, after each step it is necessary to transfer to the next apparatus to complete the subsequent step in order to establish a flow through each apparatus. Without adding fluids used to prime and clear the apparatus, some blood remains in the apparatus at each step and is lost from the system. Or if fluid is used to clear the apparatus and wash the blood into the next step then the blood becomes diluted by the fluid. Consequently, these methods add significantly to the cost of processing the transfusion blood and also reduce the amount of blood available for transfusion.

### SUMMARY OF THE INVENTION

At its most general, the present invention may provide a mass exchanger for the treatment of blood wherein the mass exchanger is at least partly collapsible. The mass exchanger may allow the composition of blood to be controlled while also being able to function as a blood bag that may for example be attached to a drip line to deliver transfusion blood directly to a patient. The collapsible nature of the mass exchanger helps to ensure that blood is delivered to the patient in a steady and continuous stream, helping to avoid for example the incorporation of air bubbles within the blood flow. Thus, the step of transferring stored blood from a mass exchanger to a blood bag may be avoided.

The collapsible nature of the mass exchanger also increases the ease of storage of the mass exchanger and helps to reduce or eliminate the amount of gas that needs to be vented before blood is introduced.

Therefore, in a first aspect, the present invention may provide a mass exchanger for use in storing blood, the mass exchanger comprising an external casing, a cavity being provided within the casing having a region for storing blood and one or more channels extending within the casing for accommodating flow of a treatment fluid, the one or more channels each being at least partly bounded by a permeable membrane to allow transfer of chemical species between the channel and the cavity; wherein the casing comprises at least one flexible wall.

In one aspect, the present invention provides a mass exchanger for use in storing blood, the mass exchanger comprising an external casing, a cavity being provided within the casing having a region for storing blood and one or more channels extending within the casing for accommodating flow of a treatment fluid, the one or more channels each being at least partly bounded by a permeable membrane to allow transfer of chemical species between the channel and the cavity; wherein the region for storing blood comprises a bag and wherein the bag comprises at least one flexible wall.

The mass transfer surface in the mass exchanger may be provided by one or more walls of the external casing or by the bag region or casing containing the blood or by one or more channels extending within the casing, or by a combination of the walls and channels within the casing. In the embodiment wherein the walls form part of the mass transfer surface, a channel is created between the external casing and the bag region so as to form a channel for accommodating the flow of a treatment fluid. The channel being bounded by a permeable membrane on the bag region side allows transfer of chemical species between the channel and the bag region, and the bag comprises at least one flexible wall.

In certain embodiments, the permeable membrane is permeable to gas, but impermeable to liquid or to ionic or dissolved species, whereas in other embodiments, the permeable membrane is microporous. A microporous membrane here is a membrane through which can pass ionic and dissolved species as well as gases, but not liquids, having pores sufficiently small to prevent leakage of liquid through the material.

For example, the membranes may be made of silicones, polymethylpentene, polyphenylene oxide or polysulphone. The membranes may also be of polyvinylchloride, which has established blood compatibility and can also be prepared with good gas permeability. The membranes may also comprise a composite material. In an embodiment the permeable membrane is used together with a rnicroporous membrane. For example, a gas permeable layer of polyvinylchloride may be backed by a thicker strengthening layer of a microporous material, Bis(2-ethylhexyl) phthalate (DEHP) is currently universally employed as a plasticiser for polyvinyl chloride in the manufacture of blood bags. However, DEHP is known to have a deleterious effect on the health of people and animals. Its use in blood bags continues because the presence of DEHP reduces the extent to which red blood cells rupture. Embodiments of the present invention are conceivable wherein the use of polyvinyl chloride plasticised with DHEP is advantageously not necessary. It is possible that by storing blood in low oxygen, similar positive effects on haemolysis can be observed without the use of DHEP. Consequently, alternative embodiments are conceivable wherein the membranes are not made of polyvinylchloride.

Typically, the mass exchanger comprises a plurality of channels.

Typically, the total surface area of the permeable membranes associated with the one or more channels is in the range of 0.05 m² to 1 m², preferably 0.05 m² to 0.5 m². With the mass transfer area being between 0.05 m² to 0.5 m² a compromise is reached between cost and the rapidity with which the blood can be brought to a desired condition. A smaller mass transfer area gives lower transfer rates but is less expensive to provide; a larger area has a higher cost but gives higher mass transfer rates so that the blood is brought to a desired condition more quickly.

In certain embodiments, the channel has a tubular shape.

In other embodiments, the channel is bounded by at least one planar membrane. For example, the channel may be provided between a pair of planar membranes that are in spaced alignment with each other.

For example, multiple channels may be provided by a set of aligned planar permeable membranes, each channel being defined by a pair of adjacent planar membranes, with blood storage spaces being provided between the channels.

In the case that the at least one channel is bounded by at least one planar membrane, the planar membrane is preferably flexible. Flexibility enables compact storage of the mass exchanger devices before and after use. It also enables the blood bags to be mounted in centrifuges used to separate blood components and for producing packed cells from whole blood. The flexible nature of the membrane facilitates filling and emptying of the blood bag without leaving an air or gas gap above the blood. In certain embodiments, the casing has an external layer that is impermeable to gas and a gas-permeable internal layer that is blood-compatible. For example, the internal layer may be plasticised PVC, or for reasons previously described, the internal layer may be one of the other gas-permeable membrane examples above. This arrangement facilitates the rapid reduction of oxygen and carbon dioxide concentrations immediately the transfusion blood (or packed cells) is introduced to the mass exchanger. This makes possible the long term maintenance of blood gas concentrations at the desired levels during storage.

Preferably, the mass exchanger comprises an indicator for providing information about the composition of blood contained within the exchanger. For example the indicator may have optical properties that vary with the composition of the blood, such as described in GB2470757. As an alternative, the indicator may comprise one or more coloured strips to indicate the desired blood colour or desired range of blood colour. During the initial treatment phase the composition of the blood due to the oxygen and carbon dioxide removal could be indicated visually and removal continued until blood gas partial pressures reached desired levels, as indicated by the indicator.

In an embodiment, the mass exchanger is also arranged to operate as a heat exchanger, wherein the treatment fluid comprises a liquid phase at a temperature, the temperature arranged so as to increase, decrease or maintain the temperature of the blood or blood product.

In a second aspect, the present invention may provide a method of storing blood, comprising the steps of:
providing a mass exchanger according to any one of the preceding claims;
introducing treatment fluid into the channel to modify the composition of the blood sample;
introducing a blood sample into the mass exchanger; and
keeping the blood sample in the mass exchanger for a storage period.

In a further aspect the steps may be carried out in a different order such that the step of introducing the treatment fluid is before the blood sample introduction or before the step of storage of the blood.

The term “blood sample” covers whole blood or blood products such as packed blood cells.

It is optimal to use donated blood as soon as possible after the donation, with the apparatus of the present invention the storage period can be extended and can be at least 24 hours. It is anticipated that, due to the possibility of controlling the composition of the sample during storage, the maximum safe storage period may be extended beyond the current limit of, for example, 42 days.

In the case of whole blood, an anti-coagulant may be incorporated into the blood or an anti-coagulant may be incorporated into the blood storage mass exchanger or blood bag.

Typically, the step of modifying the composition of the blood sample comprises modifying the concentration of oxygen and/or carbon dioxide in the sample. For example, the concentration of oxygen and/or carbon dioxide may be reduced at the start of the storage period, by passing nitrogen through the channel. The concentration of oxygen and/or carbon dioxide may be increased after the end of the storage period by passing oxygen, and/or an oxygen carbon dioxide mixture through the channel, for example before transfusion to a patient. It is thought that hyper-oxygenated blood may aid wound-healing. In certain cases, fluid may be caused to flow continuously or intermittently along the channel during the storage period, in order to maintain the concentration of oxygen and/or carbon dioxide.

An initial step may comprise the addition of carbon dioxide and the removal of oxygen in the sample. In this embodiment this may optionally be followed by a step wherein carbon dioxide and/or residual oxygen are removed. Increasing the carbon dioxide concentration within the blood greatly reduces the solubility of oxygen resulting in a very low oxygen concentration within the blood. Carbon dioxide can subsequently be easily removed from the blood due to a high mass transfer coefficient. Both oxygen and carbon dioxide levels are thus minimised within the blood.

Similarly, a blood preservative may be introduced into the blood at the start of the storage period and optionally removed after the end of the storage period. Furthermore, contaminants that may arise during storage, such as potassium ions, may be removed at the end of the storage period, and components that may become depleted, such as nitric oxide, may be introduced into the blood after the end of the storage period.

Thus, the composition of the blood sample may be modified twice: a first time through the action of a first treatment fluid e.g. in order to prepare the blood sample for storage; and a second time through the action of a second treatment e.g. in order to prepare the blood sample for transfusion to a patient.

The treatment fluid may comprise a liquid and/or a gaseous phase. In certain cases, a liquid may be caused to flow along the channel after the storage period, the temperature of the liquid being greater than the storage temperature of the blood, so as to warm the blood before a possible transfusion.

The treatment fluid may be an inert gas, such as nitrogen, to remove oxygen and carbon dioxide, it may be an oxygen rich gas to add oxygen, or may be a mixture of gases to maintain gas concentrations in the blood at desired levels. The treatment fluid may also be a gas/liquid mixture such as nitrogen and water. Where the gas-permeable membrane is non-porous, the liquid phase of the treatment fluid serves the purpose of heating or cooling the blood or of maintaining it at a desired temperature. Where the membrane is microporous, the liquid phase may be a solution with a composition such that it also adds or removes ionic or dissolved species to or from the blood, or maintains such species at desired concentrations within the blood. The treatment fluid may be changed during the treatment and storage cycle. For example, it may be an inert gas during the initial treatment phase to reduce oxygen and carbon dioxide concentrations to very low levels. During this phase, it may also contain a liquid phase to maintain the blood at a temperature to facilitate transfer of oxygen and carbon dioxide from the blood. This liquid flow may be replaced with a cold liquid flow to cool the blood rapidly prior to refrigerated storage, when the liquid flow would cease and a low flow of inert gas applied to maintain anaerobic conditions throughout the storage period. Alternatively, the treatment fluid may be replaced with a liquid with an affinity for oxygen and/or carbon dioxide that can maintain low oxygen and/or carbon dioxide concentrations in the blood without the requirement to maintain a flow of the treatment fluid.

The ability to store the blood without connection to a flow of a treatment fluid facilitates transport of the blood. Immediately prior to use, a warm liquid phase may be applied to warm the blood rapidly to transfusion temperature. At the same time an oxygen-containing gas will be employed to provide an ideal blood gas concentration for transfusion.

The treatment fluid may incorporate a gelling agent. Optionally the gelling agent may be used to cause reversible gelation of the treatment fluid. The gelling agent may be added to the treatment fluid pre- and/or post-reduction of oxygen and/or carbon dioxide levels within the blood. Such treatment may improve the handleability of a mass exchanger.

A second agent or fluid may be introduced to reverse gelation of the treatment fluid, so that flow can be re-established and an oxygenating treatment fluid can be introduced to re-oxygenate the blood.

Typically, the mass exchanger is moved at least once during the storage period in such a way as to promote circulation of the blood within it, for example, its orientation is changed. This helps to avoid the formation of pockets of untreated blood within the mass exchanger. The device may be manipulated to mix the blood and facilitate mass transfer.

In general, the method comprises the step of inspecting the blood to determine its composition. This may comprise a visual inspection, typically using one or more coloured strips or swatches for comparison with the blood colour. In other cases, the blood oxygenation may be assessed with an oximeter.

The method also comprises and envisages the use of a mass exchanger, here also referred to as a blood bag, that has a width between the walls 15 (illustrated in FIG. 2) of for example around 5 mm, that provides a sufficiently narrow width between the walls to facilitate irradiation of the blood. Irradiation can reduce or eliminate transfusion-associated graft versus host disease. It can also reduce or eliminate the risk resulting from the possible presence of harmful microorganisms in the transfused blood. An additional benefit of a device with this arrangement is the ability concurrently to remove or reduce components such as potassium ions from the blood that arise as a result of accelerated blood degradation caused by irradiation.

### BRIEF DESCRIPTION OF THE DRAWINGS

The invention will now be described by way of example with reference to the following Figures in which:

FIG. 1 shows a front view of a mass exchanger according to an embodiment of the first aspect of the invention, for use in an example of the method according to a further aspect of the invention;

FIG. 2 shows a side sectional view along a line A-A of the mass exchanger of FIG. 1 according to an embodiment of the first aspect of the invention, for use in an example of the method according to a further aspect of the invention, and

FIG. 3 shows a schematic section view of a mass exchanger according to a second embodiment of the first aspect of the invention, for use in an example of the method according to a further aspect of the invention.

### DETAILED DESCRIPTION

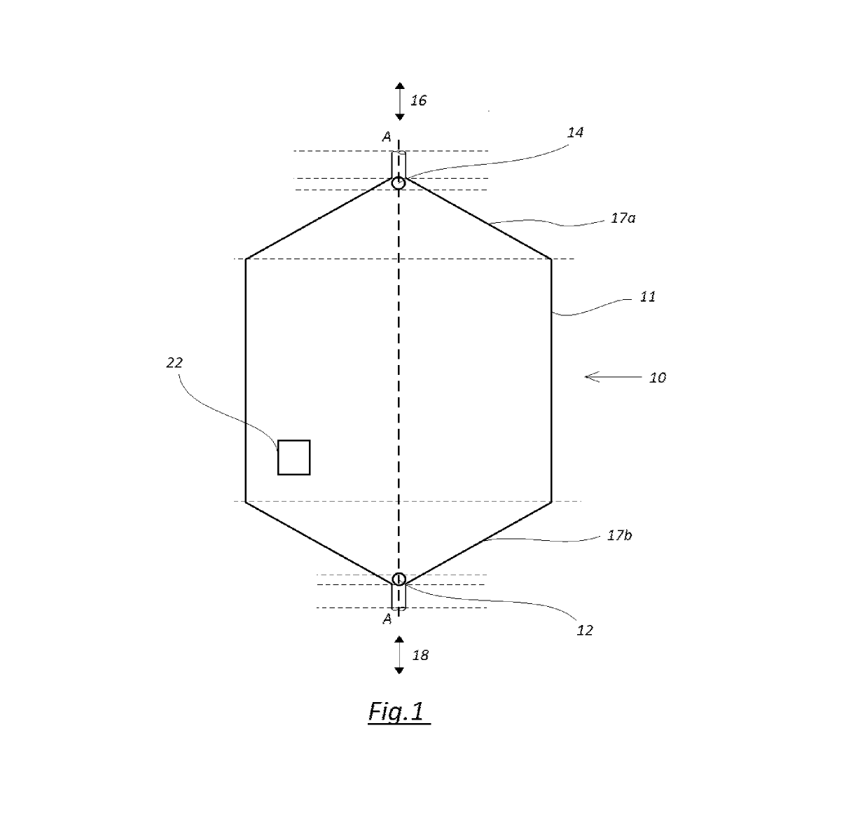

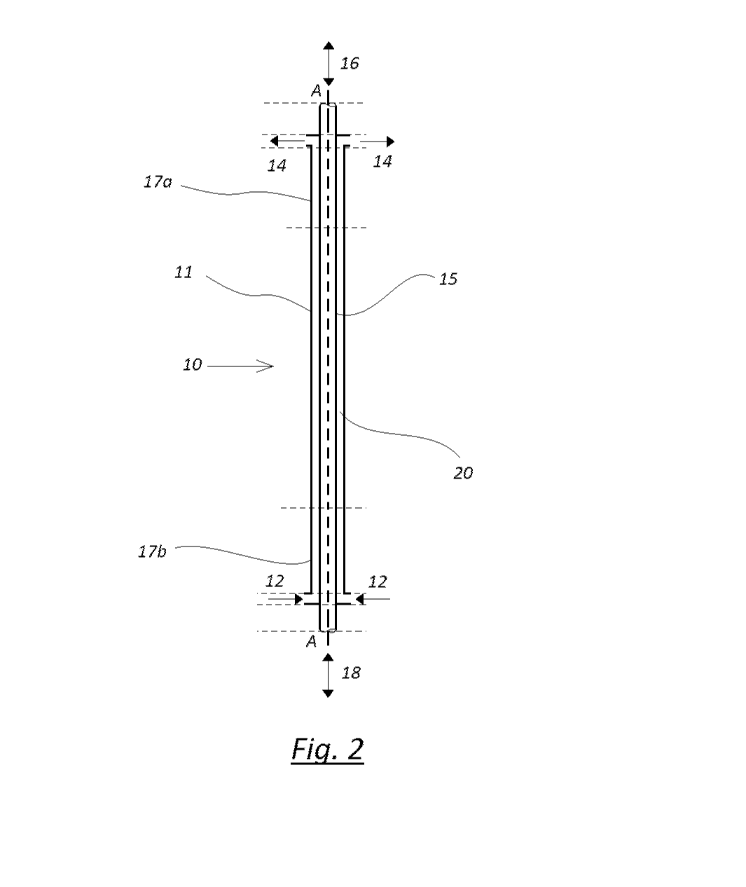

Referring to FIG. 1 and FIG. 2, a mass exchanger 10 comprises an outer casing 11 that is relatively impervious to gases. The outer casing 11 houses the storage vessel bounded by walls 15. The walls 15 may be constructed of gas-permeable polymers, such as PVC, silicones, polymethylpentene, or polyphenylene oxide, or from microporous materials such as microporous polypropylene and may be flexible. Each of the walls 15 has an area about 0.10 m² and the storage vessel or blood bag provides a mass transfer area of about 0.2 m².

Upper portions 17a and lower portions 17b of the walls of the outer casing 11 can be flexible and are sloping, providing a triangular portion (in the longitudinal direction on the page in FIG. 1). The portion of the mass exchanger 10 bounded by the gas permeable walls 15 and the outer casing 11 provides fluid communication between a treatment fluid inlet 12 and a treatment fluid outlet 14. The mass exchanger 10 is further provided with an air bleed port 16 and a blood port 18. In operation blood is fed into the device through port 18, which may also provide the blood outlet when it is subsequently transfused. Alternatively, a separate outlet port (not shown) may be provided that is similar to the ports in conventional blood bags and which is then used for the subsequent transfusion. The triangular shape with sloping walls 17a, 17b facilitate charging and discharging blood without retention of bubbles and other aspects of turbulence that could occur with other shapes of entry and exit passages. When the device is full and contains no bubbles, the ports 16 and 18 are closed. The treatment fluid is fed into ports 12 in operation (which, in practice can be joined) and flows out of ports 14 (which may also be joined), The volume of the device allows storage of one unit of blood (about 450 ml).

In the illustrated embodiment the distance between walls 15 when full of blood is about 0.005 m (5 mm). There are a number of ways envisaged of maintaining or constraining the distance between the walls 15, whilst still enabling the mass exchanger apparatus to be flattened and manipulated when not in use. A long diffusion path for the blood mass transfer will significantly reduce the efficiency of the device. A distance of around 5 mm is suitable and means that at no point in the device is there any element of blood more than a few millimetres (mm) from a permeable wall. In a similar manner, the treatment fluid channel 20 between walls 11 and permeable membrane 15 should be shaped such that no element of the treatment fluid (e.g. nitrogen) is more than a few mm from the permeable membrane. The mass transfer area may be further increased by further subdividing the volume with further channels. Alternatively, further channels may be used to maintain the total mass transfer area while decreasing the area of walls 11, 15.

In certain embodiments both the outer casing 11 and the gas permeable walls 15 are flexible.

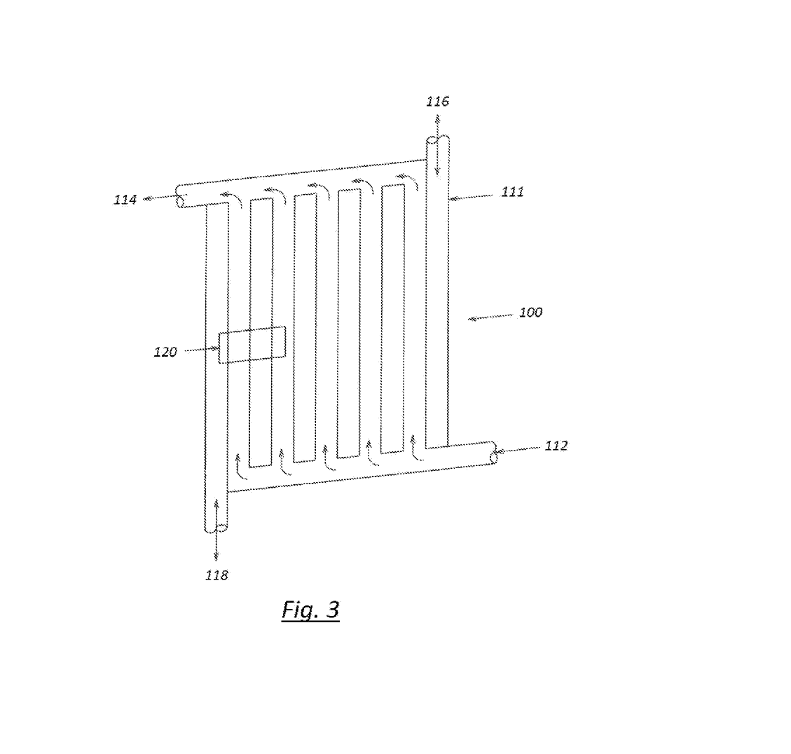

The mass transfer area may be increased by employing gas permeable tubes (hollow fibres) instead of gas permeable walls. This alternative design is discussed below with reference to FIG. 3.

Referring to FIG. 3, a mass exchanger 100 comprises a flexible casing 111 that houses a plurality of tubes. The tubes may be constructed of gas-permeable polymers, such as PVC, silicones, polymethylpentene, or polyphenylene oxide, or from microporous materials such as microporous polypropylene. Typically, the tubes provide a mass-transfer surface area of between 0.2 m² and 1 m².

The tubes provide fluid communication between a treatment fluid inlet 112 and a treatment fluid outlet 114. The mass exchanger is further provided with an air bleed port 116 and a blood port 118.

In some embodiments, a gas-permeable casing (as in FIG. 1) may be combined with gas-permeable membranes within the casing.

In certain embodiments, the flexible casing may comprise two layers: an external layer that is impermeable to gas diffusion and an internal layer that has good blood-compatibility, such as plasticised PVC or optionally, for reasons described in the summary of the invention, one of the other membrane examples provided earlier.

The casing is provided with an indicator 22 (shown in FIG. 1 as 22, not shown in FIG. 2, shown in FIG. 3 as 120) that assists in determining the composition of the blood held within the mass exchanger. The indicator 22, 120 may comprise e.g. a coloured surface for comparison with the colour of the blood within the exchanger, As an alternative, a dye layer may be incorporated into the mass exchanger, for example provided on the outer surface of a gas permeable membrane, and interrogated by suitable optical means to provide e.g. the oxygen and/or carbon dioxide partial pressures in the blood, as described in GB2470757.

In use, the mass exchanger is preferably oriented vertically with the blood port 18, 118 at the lowest point. The donated blood or blood product is fed into the vessel through connector 18, 118 and any gas in the vessel is released through vent 16, 116. When the vessel is full, or a complete unit of blood has been introduced, port 18, 118 is closed and the bag is squeezed to exhaust any remaining gas in the bag before vent 16, 116 is closed. Soon after transfer of the blood into the casing, a treatment fluid is caused to flow along the channels 20 or tubes to modify the composition of the blood so that it is better adapted for storage. The treatment fluid may comprise liquid and/or gaseous phases. For example, the treatment fluid may comprise an inert gas (for example, nitrogen), so as to reduce the concentration of oxygen and/or carbon dioxide in the blood. Alternatively, the treatment fluid may consist of a gas/liquid mixture (as described in co pending patent application PCT/GB2016/050098) which in operation can add or remove components that can diffuse through a microporous membrane. For example, it can add components to the blood that become depleted during storage or components that improve the long term storage of the blood. It can remove harmful components that accumulate during storage or as a consequence of damage caused by irradiation, and also components that may have been added to improve long-term storage.

In an alternative arrangement, the treatment fluid may be a liquid that dissolves oxygen and/or carbon dioxide either to transfer oxygen and/or carbon dioxide from the blood or to transfer oxygen and/or carbon dioxide to the blood. Examples of such liquids include solutions of porphyrins for oxygen transfer, alkaline solutions for absorption of carbon dioxide, suspensions of bound porphyrins which may be suspended in aqueous solutions that either absorb or deliver carbon dioxide, or liquids such as perfluorocarbons that dissolve both oxygen and carbon dioxide and may either deliver or absorb the gases depending on the concentration dissolved. The mass exchanger including a flexible casing which, when in combination with a liquid treatment fluid, is compatible with a centrifuge step and can provide an improvement over a flexible casing mass exchanger with a gaseous treatment fluid. The liquid treatment fluid cannot be compressed so occupies and maintains the flexible casing structure. This is particularly useful when a centrifuge step is used to separate blood components, the casing can distort when the treatment fluid is a gas and a centrifuge step is used.

An additional step may be included for assisting in achieving very low blood oxygen concentrations when blood is filling the mass exchanger storage portion. The first step involves the exchange with carbon dioxide (or a fluid with a high carbon dioxide concentration) to drive out the majority of the oxygen held in the blood product. The second step is then to effectively drive the carbon dioxide out of the blood, together with some residual oxygen, by exchange with nitrogen or another treatment fluid with a low, or zero, oxygen and carbon dioxide concentration, or with an affinity for carbon dioxide and oxygen.

The desired blood gas concentrations can be maintained by flows of gases, liquids or gas/liquid mixtures. Where a treatment fluid has sufficient affinity for oxygen and/or carbon dioxide, no flow may be needed once the desired low concentrations of the gases has been achieved. Thus, the blood bag may remain unconnected to a treatment fluid supply until it is required to restore oxygen concentration prior to transfusion.

In the same way as the use of treatment liquid in particular can have further advantages in maintaining the structural integrity of the mass exchanger then the low blood oxygen concentration step could be achieved with a flow of successive fluids between the cavities with flexible membranes. The flow cycle of treatment liquid could be, for example carbon dioxide then nitrogen to achieve low blood gas concentrations, then followed by a treatment liquid to maintain the low concentrations.

During this treatment period, the mass exchanger may be moved occasionally or regularly, for example, by changing its orientation, to help to prevent the formation of pockets of untreated blood.

During or immediately after this initial treatment period, the blood is refrigerated to an appropriate temperature for storage.

This treatment (that is, the flow of treatment fluid through the tubes and/or the movement of the mass exchanger) may be prolonged continuously or intermittently for the whole of the period during which the blood is stored, so as to maintain the composition of the blood in its treated condition. However, in certain cases (for example, when the treatment fluid is a liquid or gel with a strong affinity for oxygen) the flow of treatment fluid may cease during the storage period.

At the end of the storage period, the blood may be treated once more, to bring its composition and/or temperature closer to those required for transfusion of the blood to a recipient. For example, treatment fluid may be caused to flow along the channel or channels 20 or tubes, the properties of the treatment fluid being selected so as to achieve one or more of the following:
replenishment of oxygen (preferably until the blood is oxygenated at least to arterial conditions), carbon dioxide, and/or nitric oxide;
removal of preservative and/or removal of harmful species that may accumulate during storage such as potassium ions;
an increase in blood temperature, to bring it closer to body temperature.

Verification that the blood is ready for transfusion may be achieved by visual inspection of its colour or by means of the indicator 22,

The mass exchanger is then typically transferred to the head of a drip line for transfusion to the recipient.

1. A mass exchanger for use in storing blood or a blood product such as packed cells, the mass exchanger comprising an external casing, a cavity being provided within the casing having a region for storing blood and one or more channels extending within the casing for accommodating flow of a treatment fluid, the one or more channels each being at least partly bounded by a permeable membrane to allow transfer of chemical species between the channel and the cavity;
wherein the casing comprises at least one flexible wall.

2. A mass exchanger according to claim 1, wherein the region for storing blood comprises a bag and wherein the bag or the casing comprise at least one flexible wall.

3. A mass exchanger according to claim 1, wherein the permeable membrane is permeable to gas but impermeable to liquids or to ionic or dissolved species.

4. A mass exchanger according to claim 1, wherein the permeable membrane is microporous.

5. A mass exchanger according to claim 1, wherein the total surface area of the permeable membranes associated with the one or more channels lies in the range 0.05 m² to 1 m².

6. A mass exchanger according to claim 1, wherein at least one channel has a tubular shape.

7. A mass exchanger according to claim 1, wherein the walls of the channel comprise at least one planar membrane.

8. A mass exchanger according to claim 7, wherein the planar membrane is flexible.

9. A mass exchanger according to claim 1, wherein the casing has an external layer that is impermeable to gas and an internal gas-permeable layer that is blood-compatible.

10. A mass exchanger according to claim 1, further comprising an indicator for providing information about the composition of the blood contained within the exchanger.

11. A mass exchanger according to claim 10, wherein the optical properties of the indicator vary with the composition of the blood.

12. A mass exchanger according to claim 1, further arranged to serve as a heat exchanger, wherein the treatment fluid comprises a liquid phase at a temperature, the temperature arranged so as to increase, decrease or maintain the temperature of the blood or blood product.

13. A mass exchanger according to claim 1, wherein the treatment fluid comprises a gelling agent.

14. A mass exchanger according to claim 1, wherein the treatment fluid comprising a substance arranged to reverse gelation.

15. A method of storing blood, comprising:
providing a mass exchanger according to claim 1 any one of the preceding claims;
introducing a blood sample into the mass exchanger;
keeping the blood sample in the mass exchanger for a storage period; and
introducing treatment fluid into the channel to modify the composition of the blood sample.

16. A method of storing blood, comprising the steps of:
providing a mass exchanger according to claim 1;
introducing a blood sample into the mass exchanger;
introducing treatment fluid into the channel to modify the composition of the blood sample, and;
keeping the blood sample in the mass exchanger for a storage period.

17. A method according to claim 16, wherein the blood sample is kept within the mass exchanger for a storage period of up to or around 42 days.

18. A method according to claim 16, further comprising the step of transferring the mass exchanger after the storage period to the head of a drip line and connecting it to a supply tube for transfusing the blood sample into the patient.

19. A method according to claim 16, wherein the blood sample enters and exits the mass exchanger through the same port.

20. A method according to claim 16, wherein the mass exchanger is moved at least once during the storage period, in such a way as to promote circulation of blood within the exchanger.

21. A method according to claim 16, wherein the composition of the treatment fluid is selected so as to control the concentration of oxygen and/or carbon dioxide in the blood sample.

22. A method according to claim 21, comprising reducing the concentration of oxygen and/or carbon dioxide in the blood sample, followed by the step of increasing the concentration of oxygen and/or carbon dioxide in the blood sample after a time period up to around 42 days.

23. A method according to claim 21, further comprising causing fluid to flow continuously or intermittently along the channel for up to around 42 days, the composition of the fluid being selected to control the concentration of oxygen and/or carbon dioxide in the blood sample.

24. A method according to claim 16, wherein after the storage period, treatment fluid flows along the channel, the temperature of the treatment fluid being greater than the storage temperature of the blood.
